(19) United States
(12) Patent Application Publication
(10) Pub. No.: US 2025/0249258 A1
(43) Pub. Date: Aug. 7, 2025

(54) SYSTEMS AND METHODS FOR ADJUSTING STIMULATION BASED ON SEIZURE FREQUENCY

(71) Applicant: THE ALFRED E. MANN FOUNDATION FOR SCIENTIFIC RESEARCH, VALENCIA, CA (US)

(72) Inventors: Brian Shelton, Ventura, CA (US); Neil Talbot, La Crescenta, CA (US)

(21) Appl. No.: 19/040,802

(22) Filed: Jan. 29, 2025

Related U.S. Application Data
(60) Provisional application No. 63/548,763, filed on Feb. 1, 2024.

(51) Int. Cl. A61N 1/36; A61B 5/00; A61N 1/05

(52) U.S. Cl. CPC A61N 1/36175; A61B 5/4094; A61B 5/4836; A61B 5/686; A61N 1/0556; A61N 1/36053; A61N 1/36139

(57) ABSTRACT

A medical device includes an implantable pulse generator (IPG), and the IPG includes a driver configured to generate a stimulation current having a duty cycle; a memory storing instructions; and a microcontroller circuit operatively coupled to the driver and to the memory, and configured, in response to executing the instructions stored in the memory, to cause the driver to vary the duty cycle based at least in part by a time-variable primary duty cycle transfer function.

### CROSS-REFERENCE TO RELATED APPLICATION

This application claims priority to and the benefit of U.S. Provisional Patent Application No. 63/548,763, filed on Feb. 1, 2024, the entire content of which is hereby incorporated by reference.

### BACKGROUND

This background section is provided only for purposes of introducing certain background material relating to the present disclosure and, thus, is not an admission of prior art.

Epilepsy is a common malady that produces potentially-fatal seizures and that can be treated under appropriate circumstances with nerve stimulation, such as Vagus Nerve Stimulation (VNS), Deep Brain Stimulation (DBS), or Responsive Neurostimulation (RNS). VNS may entail the surgical implantation of a medical device into a patient's chest area under the skin to stimulate the vagus nerve with electrical impulses. The vagus nerve originates from the brainstem and traverses both sides of the neck down to the chest and abdomen. The implanted medical device may send electrical signals via the vagus nerve to the brain. A stimulation lead may connect an implantable pulse generator (IPG) of the medical device to the vagus nerve via a cuff electrode. VNS has been shown to be helpful in many cases for reducing the number and severity of seizures, particularly for patients who are less responsive to more non-invasive methods like oral medication.

The rate of seizures for epileptic patients is not constant throughout the day or from day to day. This indicates that the amount of norepinephrine in the brains of epileptic patients may vary throughout the day and over multiple days. Lower levels of norepinephrine are correlated with a higher risk of a seizure, and certain types of nerve stimulation, such as Vagus Nerve Stimulation (VNS), Deep Brain Stimulation (DBS), or Responsive Neurostimulation (RNS), can cause the body to release more norepinephrine and reduce the likelihood and severity of seizures. Accordingly, it can be beneficial to adjust the amount of electrical stimulation according to this variability in norepinephrine and risk of seizure throughout the day and over multiple days.

It is in view of this technical background that the technology of the present disclosure is presented.

### SUMMARY

This Summary section introduces some features of nonlimiting and non-exhaustive examples of the present disclosure, and is not intended to limit the scope of the claims.

According to an aspect, the technology relates to a medical device including an implantable pulse generator (IPG), the IPG including a driver configured to generate a stimulation current having a duty cycle; a memory storing instructions; and a microcontroller circuit operatively coupled to the driver and to the memory, and configured, in response to executing the instructions stored in the memory, to cause the driver to vary the duty cycle based at least in part by a time-variable primary duty cycle transfer function.

In some examples, the primary duty cycle transfer function is at least partly based on a time-variable intraday duty cycle transfer function.

In some examples, for each intraday time period of a plurality of distinct intraday time periods that collectively span a cycle period of about 24 hours, the intraday duty cycle transfer function outputs a corresponding duty cycle value during the intraday time period, wherein the intraday duty cycle transfer function is cyclical over the cycle period.

In some examples, the plurality of intraday time periods are equal to each other and include at least 4 intraday time periods.

In some examples, the intraday duty cycle transfer function is defined based at least in part on a plurality of seizure rate values respectively corresponding to the plurality of intraday time periods.

In some examples, the medical device is configured to detect seizures and to count the plurality of seizure rate values.

In some examples, the intraday duty cycle transfer function is defined based at least in part on a plurality of seizure rate values respectively corresponding to a plurality of intraday time periods that collectively span at least part of a cycle period equal to about 24 hours, each of the plurality of intraday time periods being equal to or less than 6 hours.

In some examples, the primary duty cycle transfer function is further based at least in part on a time-variable multi-day duty cycle transfer function.

In some examples, the primary duty cycle transfer function includes a scalar product of the intraday duty cycle transfer function and the multi-day duty cycle transfer function, wherein the multi-day duty cycle outputs a scalar value based on what day it is.

In some examples, the multi-day duty cycle transfer function is cyclical over a period equal to or greater than two days.

In some examples, the primary duty cycle transfer function is based at least in part on a time-variable multi-day duty cycle transfer function.

In some examples, the multi-day duty cycle transfer function is cyclical over a period equal to or greater than two days.

In some examples, the multi-day duty cycle transfer function is defined at least in part on a plurality of seizure rate values respectively corresponding to a plurality of days.

In some examples, for each day of a plurality of days, the multi-day duty cycle transfer function outputs a corresponding value.

In some examples, the output values of the multi-day duty cycle transfer function respectively for the plurality of days are based on a corresponding plurality of daily seizure rates.

In some examples, the medical device is configured to detect seizures, and to count the plurality of daily seizure rates.

In some examples, the primary duty cycle transfer function is based at least in part on a plurality of seizure rate that respectively correspond to a plurality of time periods.

In some examples, the medical device is configured to detect seizures of a person that the medical device is implanted in, and to count the plurality of seizure rates.

In some examples, the microcontroller circuit is configured to generate the duty cycle transfer function based at least in part on the plurality of seizure rate values.

In some examples, the plurality of time periods include a plurality of intraday time periods that collectively span at least part of a cycle period equal to about 24 hours.

In some examples, the plurality of intraday time periods include at least 4 intraday time periods.

In some examples, the plurality of time periods include at least two periods, each of about 24 hours.

In some examples, the microcontroller circuit is configured to time-shift, or adjust, the duty cycle transfer function.

In some examples, the primary duty cycle transfer function is based at least in part on a time-variable intraday duty cycle transfer function, and the microcontroller circuit is configured to time-shift, or adjust, the intraday duty cycle transfer function.

In some examples, the microcontroller circuit is configured to time-shift, or adjust, the intraday duty cycle transfer function based on data about at least one of a daylight savings time change, a person's sleep schedule for a previous night, the person's quality of sleep for the previous night, the person's activity schedule during the day, or the level of activity of the person during the day.

In some examples, the primary duty cycle transfer function is based at least in part on a time-variable multi-day duty cycle transfer function, and the microcontroller circuit is configured to time-shift, or adjust, the multi-day duty cycle transfer function.

In some examples, the microcontroller circuit is configured to time-shift, or adjust, the multi-day duty cycle transfer function based on data about at least one of a person's ovulation schedule or a time of year.

In some examples, the medical device includes a stimulation lead electrically coupled to the IPG; and a stimulation electrode on the stimulation lead and configured to provide electrical stimulation to tissue around the stimulation electrode, wherein the driver is configured to provide the stimulation electrode to the stimulation electrode through the stimulation lead.

In some examples, the stimulation electrode is configured to stimulate a vagus nerve.

According to another aspect, the technology relates to a medical device including an implantable pulse generator (IPG), the IPG including a driver configured to generate a stimulation current having a duty cycle; a memory storing instructions; and a microcontroller circuit operatively coupled to the driver and to the memory, and configured, in response to executing the instructions stored in the memory, to cause the driver to vary the duty cycle in accordance with a primary duty cycle transfer function that is based at least in part on a plurality of measured seizure rate values respectively corresponding to a plurality of time periods.

In some examples, the primary duty cycle transfer function includes an intraday duty cycle transfer function that outputs a duty cycle value that varies throughout a cycle period of about 24 hours.

In some examples, the plurality of time periods collectively span at least part of the cycle period, and, during each of the plurality of time periods, the intraday duty cycle transfer function outputs a duty cycle value based on the corresponding seizure rate value.

In some examples, the plurality of time periods collectively span at least part of the cycle period and include at least 4 time periods.

In some examples, the primary duty cycle transfer function includes a multi-day duty cycle transfer function that varies throughout a cycle period of two days or more.

In some examples, the plurality of time periods collectively span at least part of the cycle period and are each about 24 hours.

In some examples, during each of the plurality of time periods, the multi-day duty cycle transfer function outputs a single value based on the corresponding seizure rate value.

In some examples, the primary duty cycle transfer function includes both an intraday duty cycle transfer function and a multi-day duty cycle transfer function, the intraday duty cycle transfer function varies throughout a first cycle period of about 24 hours, and the multi-day duty cycle transfer function varies throughout a second cycle period of two days or more.

In some examples, the plurality of time periods include a first set of time periods and a second set of time periods, the first set of time periods collectively span at least part of the first cycle period and include at least 4 time periods, and the second set of time periods collectively span at least part of the second cycle period and are each about 24 hours.

In some examples, the primary duty cycle transfer function is defined at least in part by a scalar product of the intraday duty cycle transfer function and the multi-day duty cycle transfer function, wherein, for each time period of the first set of time periods, the intraday duty cycle transfer function outputs a duty cycle value during the time period based on the corresponding seizure rate value, and wherein, for each time period of the second set of time periods, the multi-day duty cycle transfer function outputs a scalar value based on the corresponding seizure rate value.

In some examples, the medical device is configured to detect seizures, and to count the plurality of seizure rate values.

In some examples, the microcontroller circuit is configured to generate the primary duty cycle transfer function based at least in part on the plurality of seizure rate values.

In some examples, the generating the duty cycle transfer function includes identifying one or more cyclical patterns in the plurality of seizure rate values with respect to their corresponding plurality of time periods.

In some examples, the microcontroller circuit is configured to time-shift, or adjust, the duty cycle transfer function.

In some examples, the microcontroller circuit is configured to time-shift, or adjust, the duty cycle transfer function based on data about at least one of a daylight savings time change, a person's sleep schedule for a previous night, the person's quality of sleep for the previous night, the person's activity schedule during the day, the level of activity of the person during the day, the person's ovulation schedule, or a time of year.

In some examples, the medical device includes a stimulation lead electrically coupled to the IPG; and a cuff electrode on the stimulation lead and configured to provide electrical stimulation to a vagus nerve, wherein the driver is configured to provide the electrical current to the cuff electrode through the stimulation lead.

According to another aspect, the technology relates to a method of operating a medical device, the medical device including an implantable pulse generator (IPG), the method including generating, by the IPG, a stimulation current having a duty cycle; and varying the duty cycle based on a plurality of seizure rate values respectively corresponding to a plurality of time periods.

In some examples, the varying the duty cycle includes varying the duty cycle over a first period of about 24 hours.

In some examples, the varying the duty cycle includes changing the duty cycle at least 4 times over the first period.

In some examples, the duty cycle is varied based on a cyclical intraday duty cycle transfer function.

In some examples, the varying the duty cycle includes varying the daily average duty cycle over a second period of two days or more, the second period including the first period.

In some examples, the plurality of time periods include at least 4 intraday time periods that collectively span at least part of the first period, and the varying the duty cycle includes, during each of the at least 4 intraday time periods, generating the stimulation current with a duty cycle that is based on the corresponding seizure rate value.

In some examples, the varying the duty cycle includes varying the daily average duty cycle over a second period of at least two days.

In some examples, the duty cycle is varied in accordance with a cyclical multi-day duty cycle transfer function.

In some examples, the plurality of time periods include at least two daylong time periods, each being about 24 hours, and the varying the duty cycle includes, for each of the at least two daylong time periods, generating the stimulation current with a duty cycle that has an average over the daylong time period that is based on the corresponding seizure rate value.

In some examples, the method includes measuring, by the IPG, the plurality of seizure rate values.

In some examples, the method includes generating, by the IPG, a duty cycle transfer function based on the plurality of seizure rate values, wherein the varying the duty cycle is based at least in part on the generated duty cycle transfer function.

In some examples, the medical device further includes a stimulation lead electrically coupled to the IPG and a cuff electrode on the stimulation lead and configured to stimulate a vagus nerve.

### BRIEF DESCRIPTION OF THE DRAWINGS

The drawings illustrate nonlimiting and non-exhaustive examples of the present disclosure.

FIG. 1 depicts a stimulation system for treating epilepsy, including an implanted medical device and an external device, according to some examples.

FIG. 2 depicts an implantable pulse generator of the implanted medical device of FIG. 1, according to some examples, together with a schematic illustration of a memory of the implantable pulse generator.

FIG. 3A depicts a stimulation lead and stimulation electrode of the implanted medical device of FIG. 1 according to some examples.

FIG. 3B depicts a stimulation lead and stimulation electrode according to some other examples.

FIG. 4A depicts seizure data of a patient according to some examples.

FIG. 4B depicts an intraday duty cycle transfer function based on the seizure data of FIG. 4A according to some examples.

FIG. 5 depicts a method for operating an implantable medical device according to some examples.

FIG. 6 depicts a method for generating an intraday duty cycle transfer function according to some examples.

FIG. 7 depicts a method for generating a multi-day duty cycle transfer function according to some examples.

### DETAILED DESCRIPTION

Nonlimiting and non-exhaustive example embodiments of systems and methods for generating a stimulation current and/or for generating duty cycle transfer functions for generating the stimulation current will be described in more detail below.

The subject matter of the present disclosure, however, may be embodied in various different forms, and should not be construed as being limited to only the illustrated and described embodiments herein. Rather, these embodiments are provided as examples so that this disclosure will be thorough and complete, and will fully convey the aspects and features of the present disclosure to those skilled in the art. Accordingly, processes, elements, and features that are not necessary to those having ordinary skill in the art for a complete understanding of the aspects and features of the present disclosure may not be described. Unless otherwise noted, like reference numerals denote like elements throughout the attached drawings and the written description, and thus, descriptions thereof will not be repeated. In the drawings, the relative sizes of elements and features may be exaggerated for clarity.

It will be understood that, although the terms “first,” “second,” “third,” etc., may be used herein to describe various elements, features, and processes, these elements, features, and processes should not be limited by these terms. These terms are used to distinguish one element, feature, or process from another element, feature or process. Thus, a first element, feature, or process described below could be termed a second element, feature, or process, without departing from the spirit and scope of the present disclosure.

It will be understood that when an element is referred to as being “on,” “connected to,” or “coupled to” another element, it can be directly on, connected to, or coupled to the other element, or one or more intervening elements may be present. In addition, it will also be understood that when an element is referred to as being “between” two elements, it can be the only element between the two elements, or one or more intervening elements may also be present.

The terminology used herein is for the purpose of describing particular embodiments only and is not intended to be limiting of the present disclosure. As used herein, the singular forms “a” and “an” are intended to include the plural forms as well, unless the context clearly indicates otherwise. It will be further understood that the terms “comprises,” “comprising,” “includes,” and “including,” when used in this specification, specify the presence of the stated elements, features, or processes, but do not preclude the presence or addition of one or more other elements, features, and/or processes. As used herein, the term “and/or” includes any and all combinations of one or more of the associated listed items. Expressions such as “at least one of,” when preceding a list of elements, modify the entire list of elements and do not modify the individual elements of the list. The use of “may” when describing embodiments of the present disclosure refers to “one or more embodiments of the present disclosure.”

As used herein, the term “about”, when used to qualify a value, includes both exactly the stated value and values within stated ranges around the stated value. As used herein, the phrase “at least part” includes part or all of the item being describe, the phrase “at least partly” includes partly or entirely or the item being described, and similar phrases have similar meanings.

Also, any numerical range recited herein is intended to include all sub-ranges of the same numerical precision subsumed within the recited range. For example, a range of “1.0 to 10.0” is intended to include all subranges between (and including) the recited minimum value of 1.0 and the recited maximum value of 10.0, that is, having a minimum value equal to or greater than 1.0 and a maximum value equal to or less than 10.0, such as, for example, 2.4 to 7.6. Any maximum numerical limitation recited herein is intended to include all lower numerical limitations subsumed therein, and any minimum numerical limitation recited in this specification is intended to include all higher numerical limitations subsumed therein. Accordingly, Applicant reserves the right to amend this specification, including the claims, to expressly recite any sub-range subsumed within the ranges expressly recited herein.

The electronic or electric devices and/or any other relevant devices or components according to embodiments of the present disclosure described herein may be implemented utilizing any suitable hardware, firmware (e.g., an application-specific integrated circuit), software, or a combination of software, firmware, and hardware. For example, the various components of these devices may be formed on one integrated circuit (IC) chip or on separate IC chips. Further, the various components of these devices may be implemented on a flexible printed circuit film, a tape carrier package (TCP), a printed circuit board (PCB), or formed on one substrate. Further, the various components of these devices may be a process or thread, running on one or more processors, in one or more computing devices, executing computer program instructions and interacting with other system components for performing the various functionalities described herein. The computer program instructions may be stored in a memory which may be implemented in a computing device using a standard memory device, such as, for example, a random-access memory (RAM). The computer program instructions may also be stored in other non-transitory computer readable media such as, for example, a CD-ROM, flash drive, or the like. Also, a person of skill in the art should recognize that the functionality of various computing devices may be combined or integrated into a single computing device, or the functionality of a particular computing device may be distributed across one or more other computing devices without departing from the spirit and scope of the example embodiments of the present disclosure.

Unless otherwise defined, all terms (including technical and scientific terms) used herein have the same meaning as commonly understood by one of ordinary skill in the art to which the present disclosure belongs. It will be further understood that terms, such as those defined in commonly used dictionaries, should be interpreted as having a meaning that is consistent with their meaning in the context of the relevant art and/or the present specification, and should not be interpreted in an idealized or overly formal sense, unless expressly so defined herein.

The detailed description set forth in connection with the appended drawings is intended as a description of various configurations and methods, and is not intended to represent the only configurations and methods in which the concepts described herein may be practiced. The detailed description includes specific details for the purpose of providing a thorough understanding of various concepts. However, it will be apparent to those skilled in the art that these concepts may be practiced without these specific details. In some instances, well known structures and components are shown schematically (e.g., block diagram form) in order to avoid obscuring such concepts.

Several aspects of embodiments according to the present disclosure will now be presented with reference to various systems and methods. These systems and methods will be described in the following detailed description and illustrated in the accompanying drawings by various blocks, components, circuits, processes, algorithms, etc. (collectively referred to as elements). These elements may be implemented using electronic hardware, computer software, or any combination thereof. Whether such elements are implemented as hardware or software depends upon the particular application and design constraints imposed on the overall system.

By way of example, an element, or any portion of an element, or any combination of elements may be implemented as a “processing system” or “controller” that includes one or more processors. Examples of processors include microprocessors, microcontrollers, graphics processing units (GPUs), central processing units (CPUs), application processors, digital signal processors (DSPs), reduced instruction set computing (RISC) processors, systems on a chip (SoC), baseband processors, field programmable gate arrays (FPGAs), programmable logic devices (PLDs), application-specific integrated circuits (ASICs), state machines, gated logic, discrete hardware circuits, and other suitable hardware configured to perform the various functionality described throughout this disclosure. One or more processors in the processing system may execute software. Software shall be construed broadly to mean instructions, instruction sets, code, code segments, program code, programs, subprograms, software components, applications, software applications, software packages, routines, subroutines, objects, executables, threads of execution, procedures, functions, etc., whether referred to as software, firmware, middleware, microcode, hardware description language, or otherwise.

Accordingly, in some examples, the functions described may be implemented in hardware, software, or any combination thereof. If implemented in software, the functions may be stored on or encoded as one or more instructions or code on a computer-readable medium. Computer-readable media includes computer storage media. Storage media may be any available media that can be accessed by a computer. For example, such computer-readable media can comprise a random-access memory (RAM), a read-only memory (ROM), an electrically erasable programmable ROM (EEPROM), optical disk storage, magnetic disk storage, other magnetic storage devices, combinations of the aforementioned types of computer-readable media, or any other medium that can be used to store computer executable code in the form of instructions or data structures that can be accessed by a computer.

Nonlimiting and non-exhaustive example systems and methods for providing stimulation will now be described in more detail with reference to the drawings.

Individuals with epilepsy, or other medical conditions associated with chronic seizures, may experience cyclical patterns in the rate at which the individual experiences seizures. These patterns may span a period of a day (i.e., 24 hours) and a multi-day period. A person's likelihood of experiencing a seizure may relate to how much norepinephrine is in the person's brain, which may vary over the course of the day and from day to day. Lower levels of norepinephrine are correlated with a higher risk of a seizure. Nerve stimulation, such as vagus nerve stimulation, can cause the body to release more norepinephrine and, thus, reduce the likelihood and severity of seizures.

This variability in norepinephrine and seizure frequency complicates attempts to prevent seizures because it changes the amount of stimulation that should be provided to adequately prevent the seizures. Additionally, the amount of stimulation that is provided to a patient should be balanced with competing concerns, such as depleting the body's bioavailability of norepinephrine (e.g., the amount of norepinephrine available to be released in response to electrical stimulation) and depleting an implanted stimulator's power source (e.g., the stimulator's non-rechargeable or rechargeable battery).

The present disclosure relates to improvements in devices and methods for providing stimulation based on a time-varying primary duty cycle transfer function and/or seizure data about the patterns of seizure rates that the patient experiences. In some examples, the primary duty cycle transfer function may be based on the seizure data. Adjusting stimulation over time in this manner can reduce the likelihood and severity of seizures over time without jeopardizing the competing concerns described above.

The primary duty cycle transfer function can be based on one or both of (1) an intraday duty cycle transfer function, which may be based on how the patient's pattern of seizure rates over the course of a day, and (2) a multi-day duty cycle transfer function, which may be based on how the patient's pattern of seizure rates over a multi-day period. The intraday and multi-day duty cycle transfer functions may be empirically based on the varying rate of seizures experienced by the patient over a day and over the multi-day period. Although the present disclosure will be primarily described with reference to providing vagus nerve stimulation, other types of nerve stimulation, such as deep brain stimulation and responsive neurostimulation to treat epileptic seizures, may be provided by the devices and methods described herein or otherwise within the scope of the present disclosure. Moreover, those skilled in the art will understand that this technology can be applied for treating other cyclically occurring medical conditions or events that are treatable via nerve stimulation.

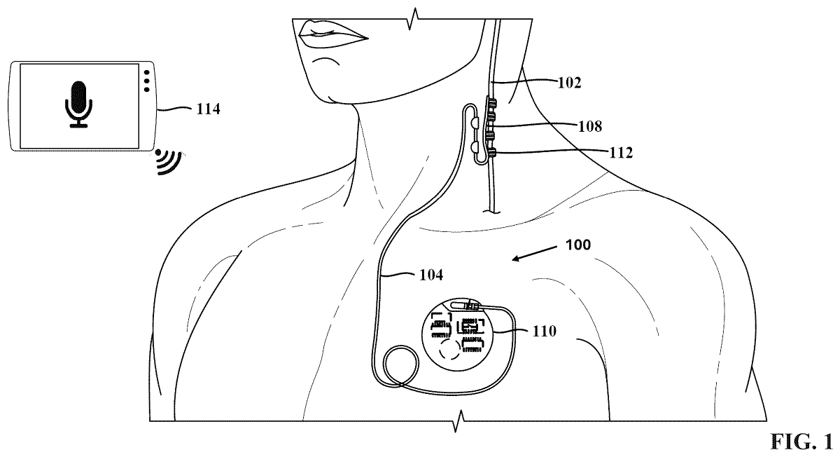

FIG. 1 depicts an example medical system that includes an implantable medical device (IMD) 100 and an external device 114 (e.g., a patient controller, a clinician programmer device, a smart phone, a smart watch, a smart ring, and/or another wearable device, etc.) that is configured to communicate with and/or control the IMD 100. The IMD 100 is depicted as being implanted under the skin of a patient. The IMD 100 may include an implantable pulse generator (IPG) 110, a stimulation lead 104 electrically coupled to the IPG 110, and a stimulation electrode 108 on the stimulation lead 104. In the example depicted, the stimulation electrode 108 is a cuff electrode that is configured to stimulate the vagus nerve 102. However, other types of stimulation electrodes having different configurations are also within the scope of the present disclosure.

The cuff electrode 108 may, in some embodiments, include four electrode pairs 112 that are configured to contact the vagus nerve 102. The electrodes 112 may be selectively activated, for example, to provide and/or identify acceptable amplitudes for the different cathodes. In some embodiments, single electrodes are used. In other embodiments, pairs of electrodes are used. The stimulation of the vagus nerve using electrical pulses is believed to stabilize abnormal electrical activity in the brain which can lead to seizures. For example, stimulating the vagus nerve can cause the body to release more norepinephrine, which can reduce the likelihood and severity of a seizure. The IPG 110 may also include a rechargeable or a primary cell, non-rechargeable battery for transmitting an electrical stimulation current (e.g., electrical pulses) using a selectable pulse width and frequency. The IPG 110 may further include an outlet portion for enabling a connection between the IPG 110 and the stimulation lead 104, which enables current to flow from the IPG 110 to the vagus nerve 102 via the cuff 108 and electrode(s) 112.

The IPG 110 may be configured to control (e.g., via a controller 220) various stimulation parameters of a stimulation current generated by the IPG 110 and provided to the cuff 108 and electrodes 112. These stimulation parameters may include pulse amplitude, pulse width, pulse frequency, duty cycle, ramp-on rate, and/or ramp-off rate. The ramp-on-rate is the rate of descent of a few beginning pulses in the train of pulses ramping from zero pulse amplitude to the programmed pulse amplitude in the train. In some aspects, when stimulation is applied by the IMD 100 (e.g., when the IPG 110 generates the stimulation current), there will be a stimulation period T₁ of a train of stimulation pulses, the stimulation pulses being provided with a pulse amplitude, pulse width, and pulse frequency. Then a period of no stimulation T₂ (referred to as a quiescent period) may follow the stimulation period T₁. This cycle (e.g., cycles of pairs of the stimulation period T₁ and the quiescent period T₂) may repeat any number of times, continuing until stimulation is terminated by a controller 220 (e.g., a microcontroller circuit) of the IPG 110. The “duty cycle” of stimulation (e.g., of the stimulation current) may be calculated using T₁ and T₂, and may be expressed as a fraction (duty cycle=T₁/(T₁+T₂)) or alternatively as a percentage (duty cycle=(100×T₁)/(T₁+T₂)). Thus, references to the term “duty cycle” herein should be understood as references to a parameter as defined by the foregoing calculations.

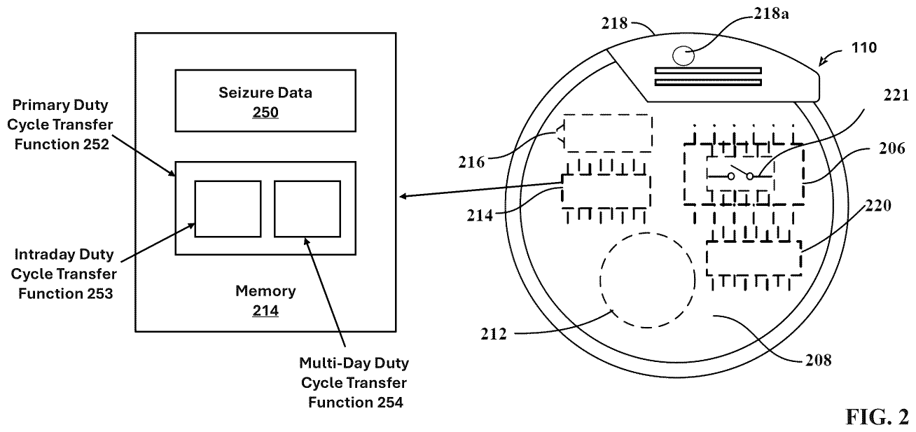

FIG. 2 depicts the IPG 110 of the IMD 100 of FIG. 1, according to some examples, together with a schematic illustration of a memory 214 of the IPG 110. The IPG 110 may include the controller 220 with processing circuitry configured to control (e.g., controllably modulate or controllably adjust) at least one stimulation parameter (e.g., pulse amplitude, pulse width, pulse frequency, duty cycle, ramp-on rate, and/or ramp-off rate) of the electrical stimulation pulses generated by the IPG 110. For example, the controller 220 may be configured to controllably adjust the duty cycle of the stimulation pulses provided, through the stimulation lead 104, to the cuff 108 and electrodes 112, as explained in more detail below. The controller 220 may be configured to maintain one or more other stimulation parameters, such as the pulse amplitude, pulse width, and/or pulse frequency, while it controllably adjusts the duty cycle. It can be desirable to maintain the pulse amplitude, pulse width, and pulse frequency, which can control how deeply into a nerve bundle (e.g., the vagus nerve) the stimulation pulses penetrates and which nerve fibers and types of nerve fibers are activated by the stimulation pulses. It can be desirable to repeatedly target the same nerve fibers for reliable and reproducible stimulation treatment.

The IPG 110 may further comprise a driver 206 which may be operatively coupled to the controller 220 and configured (e.g., programmed) to generate a periodic electrical pulse having a set pulse frequency and pulse width. For example, the driver 206 may be configured to generate the stimulation current based on control signals received from the controller 220. The driver 206 may be battery-powered and can be activated and deactivated (the latter causing the current to be turned off) via a switch 221. The connections on the integrated circuits of the IPG 110 may be coupled together selectively via a small printed circuit board 208. In other embodiments, the IPG 110 may be implemented as an SoC on a die, or a packaged die.

The IPG 110 may include a transceiver 216 (e.g., a transmitter and a receiver, which may be separate or integrally combined into one device). In some embodiments, the transceiver 216 includes a wireless receiver configured to receive wireless signals, e.g., Bluetooth Low Energy, from a source external to the patient (e.g., for communication with an external device, such as the external device 114, or an implanted electronic device). In some embodiments, the transceiver 216 may further comprise a wireless transmitter, e.g., for providing feedback to an external device, such as the external device 114, or to an external sensor. In still other embodiments, the transceiver 216 may be connected by a wire or wirelessly to a sensor for receiving information from the vagus nerve or another part of the body. The wireless receiver in transceiver 216 may be configured in some embodiments to receive (e.g., from the external device 114) instructions or other data as described herein, for the controller 220 to modulate one or more parameters of the electrical stimulation pulses. When acting as a transmitter, the transceiver 216 can provide feedback signals to external sources and devices (e.g., to the external device 114) using data generated by controller 220.

In some embodiments, information received from the wireless transceiver 216 may be stored in a memory 214. The controller 220 may be coupled to the memory 214 and configured to access the memory 214 to receive and process instructions to modulate one or more parameters of the stimulation pulses, or to temporarily deactivate the driver 206. For example, the controller 220 may be configured to deactivate the driver 206 (e.g., by opening the switch 221) during the quiescent period T₂.

More generally, the controller 220 may be operatively coupled to other elements of the IPG 110, and the memory 214 may store instructions that, in response to being executed by the controller 220, cause the controller 220 to control such elements to perform certain operations and processes described herein or otherwise within the scope of the present disclosure. The memory 214 may store instructions that cause the controller 220 to cause the IPG 110 to perform any operations and methods described herein.

The driver 206 may generate the stimulation current (e.g., stimulation pulses), which may be provided to an outlet portion 218 to which the stimulation lead 104 is attached. In some embodiments, the outlet portion 218 may include an aperture 218a for a lead connector to be inserted to attach the stimulation lead 104.

In some examples, the IPG 110 includes an internal clock 212, and the IPG 110 may be configured to measure and keep track of the time of day and/or what day it is via the internal clock 212. The controller 220 may be configured to control the stimulation current generated by the driver 206 based on seizure data 250 and/or a primary duty cycle transfer function 252, and in view of the time of day and/or what day it is, according to the internal clock 212.

While various configurations and operations of the IPG 110 have been described, it will be appreciated by those skilled in the art upon review of this disclosure that different configurations and operations may be used. For example, the controller 220 may include more than one integrated circuit, or it may include a separate module. The controller 220 may further include one or more general purpose processors, RISC processors, or other types of processors. The controller 220 may in some embodiments include dedicated hardware. For example, the controller 220 may be any one or more of a digital signal processor (DSP), a field programmable gate array (FPGA), an application-specific integrated circuit (ASIC), or a combination of digital logic devices.

The memory 214 may include any suitable memory, such as a combination of volatile and non-volatile memory, dynamic random access memory (DRAM), static random access memory (SRAM), read only memory, flash or other solid state memory, or the like. Other types of memory are possible. Non-volatile memory within the memory 214 may be used to store critical settings to enable system reset, for example. Pulse frequency, pulse amplitude, and/or pulse width values may be stored in the memory 214. The seizure data 250 and the primary duty cycle transfer function 252 may be stored in the memory 214. In some embodiments, the memory 214 may be accessible and programmable as noted above via the controller 220, or via data or instructions received via wireless receiver 216. The memory 214 may also include firmware for use by the controller, or other program information for automatically modulating one or more parameters of the electrical pulses. It will be appreciated that the IPG 110 need not be circular or elliptical in nature, and may take on different shapes based on different design considerations and patient needs. More generally, the components identified in the various figures may take on different geometries than those shown.

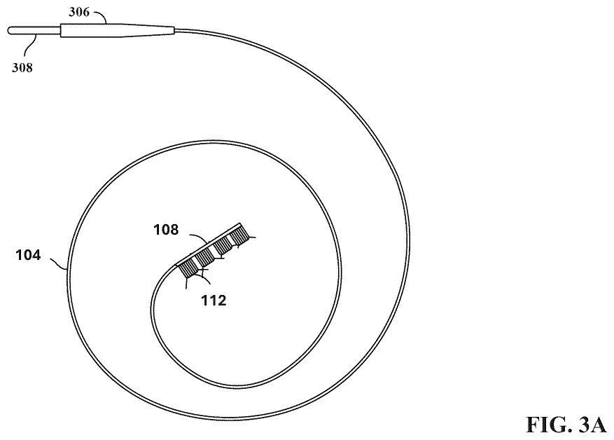

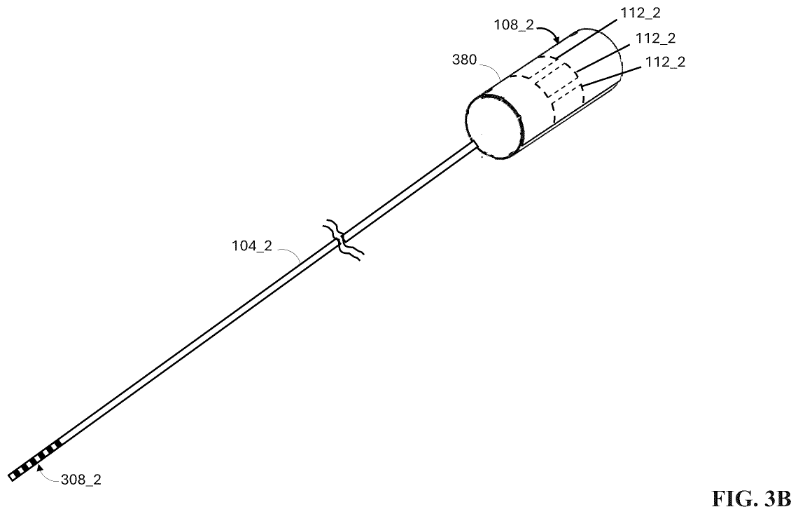

FIG. 3A depicts the stimulation lead 104 and cuff electrode 108 of the IMD 100 of FIG. 1 according to some examples. A lead connector 308 may be inserted into the IPG 110, for example, into the aperture 218a. It will be appreciated that the components may not be drawn to scale, and typically the lead connector 308 in this embodiment would be sized very small, and/or angled differently, to be minimally intrusive to the patient in which the component is implanted. In some embodiments, an insulating sheath 306 may provide support for the lead connector 308 as the insulated stimulation lead 104 terminates at the cuff electrode 108.

FIG. 3B depicts a stimulation lead 104_2 and nerve cuff electrode 108_2 according to some other examples. The stimulation lead 104_2 and nerve cuff electrode 108_2 may be used with the IMD 100 of FIG. 1 instead of the stimulation lead 104 and stimulation electrode 108 in some examples. The stimulation lead 104_2 may include, at a proximal end, a lead connector 308_2 configured to be inserted into the IPG 110, for example, into the aperture 218a. The stimulation lead 104_2 may include, at a distal end, the nerve cuff electrode 108_2. The nerve cuff electrode 108_2 may have a flexible body 380 that is configured to wrap around a nerve. The nerve cuff electrode 108_2 may include a plurality (e.g., 3, 4, 6, 8, or more) of electrode contacts 112_2 on the flexible body 380 and that are arranged to at least partially circumferentially surround the nerve when the flexible body 380 is wrapped around the nerve. The plurality of electrode contacts 112_2 may be electrically coupled (e.g., separately electrically coupled) to a respective plurality of terminals on the lead connector 308_2.

Non-limiting and non-exhaustive operations of the IMD 100 will now be described, which may include providing, by the driver 206 and under the control of the controller 220, an electrical stimulation current with a time-varying duty cycle. The stimulation current may include a plurality of pulses and have a stimulation period T₁ and a quiescent period T₂, as described herein with reference to the driver 206. The time-varying duty cycle may be based at least in part on the time-variable primary duty cycle transfer function 252 and/or the seizure data 250. The seizure data 250 may include data relating to the number of seizures (or seizure rate) that a patient experiences over time. The primary duty cycle transfer function 252 and the seizure data 250 may be stored in the memory 214.

As explained above, the rate at which a patient experiences seizures may vary over the course of a day and/or over the course of a cyclical multi-day period. The likelihood and severity of seizures can be reduced based on how much electrical stimulation is provided to the vagus nerve. Accordingly, the IMD 100 may be configured to provide a varying amount of electrical stimulation over the course of the day and/or over the multi-day period. This allows the IMD 100 to strategically increase the amount of stimulation at times and/or days in which the patient is expected to experience more seizures and to decrease the amount of stimulation at times and/or days in which the patient is expected to experience fewer seizures. The IMD 100 can therefore improve seizure treatment without sacrificing other important considerations, such as not depleting the patient's bioavailability of norepinephrine and the IMD's power source (e.g., battery).

The amount of stimulation can be controlled by varying one or more stimulation parameters, such as pulse amplitude, pulse width, pulse frequency, and duty cycle. However, pulse amplitude, pulse width, and pulse frequency may affect which nerve fibers in the vagus nerve (or other nerve bundle being stimulated) are targeted when the stimulation current is provided. Treatment can be more effective and reliable when the same nerve fibers are repeatedly targeted during stimulation. On the other hand, the amount of stimulation can also be varied by changing the duty cycle, which generally will not affect which nerve fibers are targeted. Accordingly, embodiments where the IMD 100 is configured to controllably adjust the duty cycle over time will be primarily described below. In some such embodiments, the pulse amplitude, pulse width, and/or pulse frequency may be kept constant while the duty cycle is varied. In some other embodiments, the pulse amplitude, pulse width, and/or pulse frequency are varied over time while the duty cycle is varied.

The duty cycle may be varied based on the seizure data 250, which may include data relating to the number of seizures that the patient experiences over the course of a day (e.g., during each intraday period of a plurality of intraday periods spanning about a day) and/or over the course of the multi-day period (e.g., during each period of about a day over the course of the multi-day period). The number of seizures may be empirically obtained, for example, prior to applying treatment. In some other examples, the number of seizures may be obtained while the patient is receiving stimulation treatment.

The number of seizures may be detected and counted by the IMD 100 or by another device for detecting and counting seizures in a patient. For example, the IMD 100 may include one or more sensors configured to detect the occurrence of a seizure, and the IMD 100 may be configured (e.g., via the controller 220) to count the number of detected seizures and store data relating to the counted number of seizures in the memory 214. For example, the IMD 100 may count a number of seizures experienced during each intraday period or each period of about a day. In some other examples, a separate sensor device may be used to detect and count the number of seizures in the patient over time, and the seizure data 250 may be transmitted to the IMD 100.

The IMD 100 may be configured to vary the duty cycle over time based on the time-variable primary duty cycle transfer function 252. The primary duty cycle transfer function 252 may be based on (e.g., generated based on) the seizure data 250. For example, the primary duty cycle transfer function 252 may be a function that outputs a duty cycle value based on what day it is and/or what time it is, and the seizure rate that the patient is expected to experience at said day and/or time in view of the seizure data 250. The primary duty cycle transfer function 252 may be generated by the IMD 100 (e.g., by the controller 220) or by a separate device (e.g., by the external device 114 or another computing device) and then transmitted to the IMD 100. The controller 220 may be configured to access the seizure data 250 and/or the primary duty cycle transfer function 252 and to control the driver 206 (e.g., control the duty cycle of the stimulation current generated by the driver 206) based on the seizure data 250 and/or the primary duty cycle transfer function 252.

The primary duty cycle transfer function 252 may be based at least partly on a time-variable intraday duty cycle transfer function 253 and/or a time-variable multi-day duty cycle transfer function 254. The time-variable intraday duty cycle transfer function 253 may vary the duty cycle throughout the course of a day, while the multi-day duty cycle transfer function 254 may vary the duty cycle over the course of a multi-day period.

The intraday duty cycle transfer function 253 may output a duty cycle value based on the time of day and may be generated based on the number of seizures that the patient experiences over the course of a day. The intraday duty cycle transfer function 253 may be cyclical over a first cycle period of about 24 hours (e.g., within 5%, 1%, or 0.1% of exactly 24 hours). Many patients may experience a pattern of seizure rates that is generally repeatable over exactly 24 hours. However, it's possible for some other patients to experience patterns of seizure rates that deviate from being cyclical over exactly 24 hours, and the first cycle period may be set accordingly.

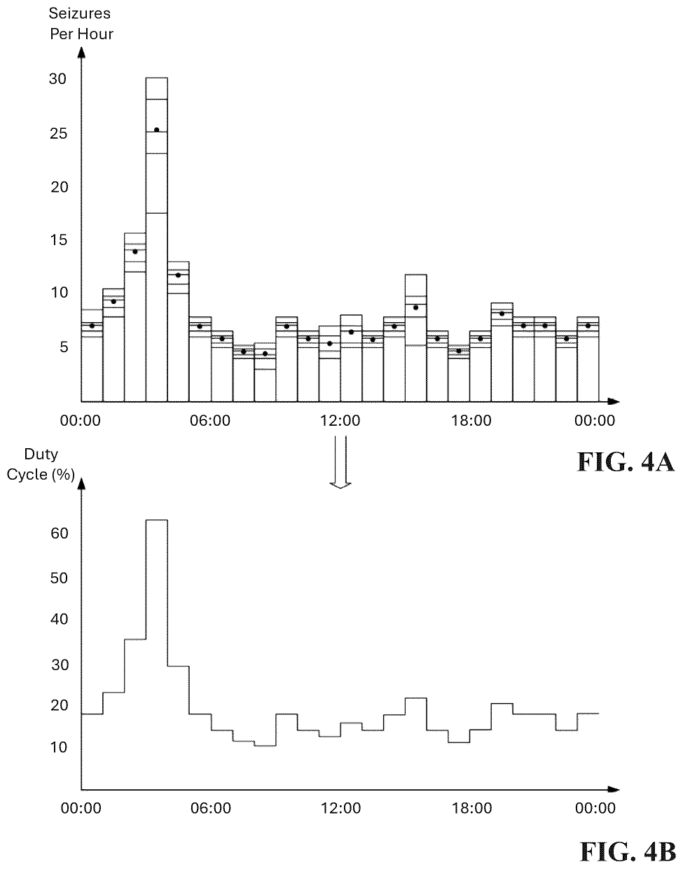

FIG. 4A depicts data (a bar graph) relating to the number of seizures experienced by a patient during each hour of the day, from midnight to midnight, for each of five days, where the five measurements for a particular hour are represented by a bar with five horizontal lines (including the line defining the top of the bar) that represent the five measurements for that hour. A dark dot is provided in each bar to represent the median number of seizures. The data in FIG. 4A indicates that the patient experiences a significant spike in seizures around 3-4 AM and experiences lesser variability in seizures over the rest of the day. FIG. 4B depicts an intraday duty cycle transfer function that is based on the data from FIG. 4A. The intraday duty cycle transfer function of FIG. 4B has 24 outputs corresponding to the 24 one-hour periods of FIG. 4A. Similar to FIG. 4A, the output of the intraday duty cycle transfer function of FIG. 4B significantly increases around 3-4 am and varies to a lesser degree over the remainder of the day.

The intraday duty cycle transfer function of FIG. 4B outputs a constant duty cycle value during each of the 24 one-hour periods and is cyclical over a period of exactly 24 hours. However, the present disclosure is not limited thereto.

In some other examples, the intraday duty cycle transfer function 253 is cyclical over a first cycle period of about 24 hours (e.g., within 5%, 1%, or 0.1% of exactly 24 hours).

The first cycle period may include a plurality of distinct intraday time periods that collectively span at least part of the first cycle period. The intraday duty cycle transfer function 253 (in FIG. 2) may output, for each intraday time period, a corresponding duty cycle value based on the seizure data 250, for example, based on the number of seizures experienced during the intraday time period. The outputs corresponding to the plurality of intraday time periods may generally be different from each other. For example, at least two of the outputs may be different from each other. The IMD 100 may be configured to generate the intraday duty cycle transfer function 253 to differentially output duty cycle values based on the time of day, for example, in accordance with the corresponding number of seizures in the seizure data 250 for that time or time period.

The plurality of intraday time periods may be equal to each other or of diverse lengths of time. In some examples, the plurality of intraday time periods includes at least 3, 4, 6, 8, 12, 24, 48, or 96 time periods. Each of the plurality of distinct intraday time periods may be less than 8 hours, 6 hours, 4 hours, 2 hours, 1 hour, 30 minutes, or 15 minutes.

In some examples, the output of the intraday duty cycle transfer function 253 varies continuously (and, in some examples, smoothly) over at least part of the first cycle period and based on the seizure rate data 250 that the intraday duty cycle transfer function 253 was generated based on. For example, generating the intraday duty cycle transfer function 253 may include generating or fitting a curve based on the seizure rate data 250 such that the curve varies continuously (and, in some examples, smoothly) over at least part of the first cycle period.

In some examples, the first cycle period is determined (e.g., by the controller 220 or a person, such as a physician) by analyzing the seizure rate data 250 and identifying (e.g., automatically via an algorithm or manually by a person) a generally cyclical pattern among seizure rates over a plurality of days (e.g., over a plurality of sequential days) and setting the first cycle period based on the identified cyclical pattern. For example, the first cycle period may be set to be equal to the length of time (e.g., average or median length of time) of the identified cyclical pattern.

The multi-day duty cycle transfer function 254 may be cyclical over a second period of two days or more (e.g., at least 3, 5, 7, 10, 20, or 30 days). The primary duty cycle transfer function 252 may be based on (e.g., may include) the multi-day duty cycle transfer function 254 with or without the intraday duty cycle transfer function 253. Some patients may not only experience repeatable changes in the rate of seizures over the course of a day but also over the course of a multi-day cycle, where the patient predictably experiences a higher number of seizures during certain day(s) of the multi-day cycle and a fewer number of seizures during other day(s) of the multi-day cycle.

The multi-day duty cycle transfer function 254 can therefore be used to adjust treatment of the patient to account for this repeating pattern in a similar way as the intraday duty cycle transfer function 253 can adjust treatment to account for a repeating pattern in seizure rates over the course of a day.

The multi-day duty cycle transfer function 254 may be generated (e.g., by the controller 220) based on the seizure data 250, which may include empirical data relating to the number of seizures the patient has over a plurality of days (e.g., a plurality of sequential days). In some examples, the patient does (or does not) receive stimulation treatment during the plurality of days. Seizures may be detected and counted in the patient for each day of the plurality of days.

The controller 220 (or other computing device) may be configured to analyze the seizure data 250 and identify (e.g., via an algorithm) a generally cyclical pattern among the seizure rate data. This may be done, for example, by autocorrelation of the daily seizure counts, by identifying the number of days that result in a minimum error from one period to the next, and/or by aligning the minima and/or maxima of the seizure counts. The controller 220 may then set the second cycle period based on the identified cyclical pattern. For example, the controller 220 may set the second cycle period to be equal to the length of time (e.g., average or median length of time) of the identified cyclical pattern. The controller 220 may be configured to generate the multi-day duty cycle transfer function 254 based on the seizure rate data 250 in each cycle covered by the plurality of days for which seizure data 250 was obtained. For example, the seizure data 250 may include data for a plurality of days that spans a plurality of the second cycle periods. In some examples, the controller 220 may be configured to calculate, from the seizure data 250, an average or median seizure rate value for each day in the second cycle period, and the controller 220 may be configured to generate the multi-day duty cycle transfer function 254 based on the average or median seizure rate values in a manner similar to how the intraday duty cycle transfer function 253 may be generated based on intraday seizure rate data.

In some examples, such as when the multi-day duty cycle transfer function 254 is used without the intraday duty cycle transfer function 253, the multi-day duty cycle transfer function 254 may output a duty cycle value based on what day within the second cycle period it is. For example, the multi-day duty cycle transfer function 254 may output a single duty cycle value over the course of each day. In some other examples, such as when the multi-day duty cycle transfer function 254 is used together with the intraday duty cycle transfer function 253, the multi-day duty cycle transfer function 254 may output a value (e.g., a scalar value) to adjust the primary duty cycle transfer function 250 based on what day within the second cycle period it is. For example, the primary duty cycle transfer function 250 may include the scalar product of the intraday duty cycle transfer function 253 and the multi-day duty cycle transfer function 254, the intraday duty cycle transfer function 253 may output a duty cycle value based on what time it is, and the multi-day duty cycle transfer function 254 may output a scalar value based on what day within the second cycle period it is in order to adjust the duty cycle value output by the intraday duty cycle transfer function 253.

In some examples, the multi-day duty cycle transfer function 254 may output, for each day in the second cycle period, a corresponding value (e.g., a duty cycle value or a scalar value) for that day. The output may be constant over the course of that day. The output may be based on the seizure data 250, for example, based on the corresponding number of seizures experienced on that day. The outputs corresponding to the plurality of days in the second cycle period may generally be different from each other. For example, at least two of the outputs corresponding to two different days may be different from each other. The controller 220 may be configured to generate the multi-day duty cycle transfer function 254 to differentially output a value based on what day within the second cycle period it is, for example, in accordance with the corresponding seizure rate data.

In some examples, the multi-day duty cycle transfer function 254 may output a single value over the course of each sub-period of a plurality of sub-periods spanning at least part of the second cycle period. A sub-period of the plurality of sub-periods may be equal to about 24 hours (e.g., within 5%, 1%, or 0.1% of exactly 24 hours). In some examples, the sub-period is equal to the first cycle period.

In some examples, the output of the multi-day duty cycle transfer function 254 changes continuously (and smoothly, in some examples) over time (including within a single day or sub-period) for at least part of the second cycle period and based on the corresponding seizure rate data. For example, the generating the multi-day duty cycle transfer function 254 may include generating or fitting a curve based on the seizure rate data such that the curve varies continuously (and smoothly, in some examples) over at least part of the second cycle period.

In some examples, the intraday duty cycle transfer function 253 and/or the multi-day duty cycle transfer function 254 may be adjusted (e.g., by the controller 220 or manually, for example, by the patient or physician) after these duty cycle transfer functions are initially generated. As discussed above, the intraday and multi-day duty cycle transfer functions may be generated based on empirical seizure rate data collected during a period of time when the patient may not have been receiving stimulation treatment. Seizure rate data may be collected (e.g., by the IMD or another seizure sensor device or method) after the intraday and multi-day duty cycle transfer functions are initially generated and one or both of the intraday and multi-day duty cycle transfer functions may be adjusted based on the subsequently obtained seizure rate data. This may allow for one or both of the intraday and multi-day duty cycle transfer functions 253 and 254 to be fine-tuned in order to continue to improve the patient's treatment.

For example, if it is determined that the patient experiences seizures at a higher rate during certain hour(s) of the day compared to the rest of the day, then the intraday duty cycle transfer function 253 may be adjusted to increase the output during those identified hour(s) of the day. This may flatten out the rate of seizures experienced by the patient over the course of the day. The controller 220 may be configured to adjust the intraday duty cycle transfer function based on subsequently obtained seizure rate data (e.g., seizure rate data obtained while the patient is receiving the stimulation treatment) to reduce variability in the seizure rates experienced by the patient over the course of the day. For example, the controller 220 may be configured to adjust the intraday duty cycle based on the variability of the subsequently obtained seizure rate data.

Similarly, if it is determined that the patient experiences seizures at a higher rate during certain day(s) within the second cycle period, then the multi-day duty cycle transfer function 254 may be adjusted to increase the output for those day(s). The controller 220 may be configured to adjust the multi-day duty cycle transfer function 254 based on subsequently obtained seizure rate data (e.g., seizure rate data obtained while the patient is receiving the stimulation treatment) to reduce variability in the seizure rates experienced by the patient over the course of the day. In some other examples, this adjustment may be entered manually, for example, by the patient or physician. For example, the controller 220 may be configured to adjust the multi-day duty cycle transfer function based on the variability of the subsequently obtained seizure rate data.

One or both of the intraday and multi-day duty cycle transfer functions 253 and 254 may be adjusted (e.g., by the controller 220 or manually, for example, by the patient or physician) based on the subsequently obtained seizure rate data to reduce the total number of seizures experienced by the patient. For example, after the seizure rates have been generally flattened out over the first and second cycle periods by fine-tuning the intraday and multi-day duty cycle transfer functions, one or both of the intraday and multi-day duty cycle transfer functions may be adjusted so that their respective outputs are increased (e.g., uniformly increased) over the first and second cycle periods. This can reduce the total number of seizures experienced by the patient without increasing the variability of the seizure rates over the first and second cycle periods.

A patient's seizures may depend on various factors, such as his or her biology, environment, and/or personal activities. When these factors are generally constant, the patient's patterns of seizures may reliably cycle through the first and second cycle periods. However, if the patient experiences changes in one or more of these factors, then the patient's patterns of seizures may be disturbed, and the controller 220 may be configured to adjust one or both of the intraday and multi-day duty cycle transfer functions 253 and 254 based on these changes. In some other examples, this adjustment may be entered manually, for example, by the patient or physician. In some examples, such changes may cause the patient to suddenly be further along, or farther behind, in the first or second cycle periods than where the patient would be expected to be based on the time of day and calendar date, and one or both of the intraday and multi-day duty cycle transfer functions 253 and 254 may be adjusted accordingly.

The controller 220 may be configured to adjust one or both of the intraday and multi-day duty cycle transfer functions 253 and 254 based on (e.g., to account for) changes in these factors. The IMD 100 may be configured to monitor (e.g., to detect changes in) changes in one or more of these factors and/or the IMD 100 may be configured to receive (e.g., from the external device 114) information about changes in one or more of these factors. Activity factors may include the patient's sleep schedule, the patient's quality of sleep, the person's activity (e.g., work or exercise) schedule, and the patient's activity level (e.g., mild, moderate, or vigorous workout), each of which may influence the patient's susceptibility to seizures. Environmental factors may include changes resulting from daylight savings time changes, which may push the patient's biological clock forward or backward by an hour compared to the time of day, and the time of year, which may directly or indirectly affect the patient's susceptibility to seizures due to, for example, changes in weather conditions and hours of sunlight. Biological factors may include a woman's ovulation schedule, which may affect her susceptibility to seizures.

The intraday and multi-day duty cycle transfer functions 253 and 254 may be adjusted based on changes in one or more of these factors by, for example, time-shifting the duty cycle transfer function and/or adjusting (e.g., differentially adjusting) the outputs of the duty cycle transfer function.

For example, the intraday pattern of seizure rates may be closely tied to when the patient wakes up and goes to bed. If the patient shifts the time that he or she wakes up at forward or backwards, then the controller 220 may be configured to time-shift the entire intraday duty cycle transfer function 253 forward or backwards based on when the patient woke up (e.g., based on the difference between the patient's expected wake-up time and the patient's actual wake-up time). The IMD 100 may be configured to detect when the patient wakes up, or the IMD 100 may receive (e.g., from the external device 114) about the time at which the patient woke up.

The patient's intraday pattern of seizure may also be affected by the patient's sleep quality. For example, a night of poor sleep quality may result in the patient having an elevated seizure rate during the morning hours or throughout the entire day. If the patient experiences poor sleep quality during one night, then the next morning the patient can transmit (e.g., via a controller) information about his or her poor sleep to the IMD 100, and the controller 220 may be configured to adjust (e.g., increase) the outputs of the intraday duty cycle transfer function 253 for the whole day or only for select time(s) in the morning to compensate for the expected increase in seizure rates. In some examples, the IMD 100 may be configured to measure the patient's sleep quality, and the controller 220 may be configured to automatically adjust the intraday duty cycle transfer function 253 based on the measured data.

The patient may experience reduced seizure rates while engaged in physical activities (e.g., exercising or working), and the intraday duty cycle transfer function 253 may initially output decreased duty cycle values during a time period when the patient normally is engaged in physical activity during the day. If the patient shifts his or her time for engaging in such activity forward or backwards by a set time period, then the controller 220 may be configured to time-shift the intraday duty cycle transfer function 253 based on the change in time, or to adjust the outputs of the intraday duty cycle transfer function for select time(s) when the patient is expected to be exercising and not exercising. The patient may transmit information about his or her activity schedule to the IMD 100, for example, via the external device 114. In some examples, the IMD 100 may be configured to detect the patient's activity level (e.g., based on the patient's measured heart rate and/or blood pressure), and the controller 220 may be configured to adjust the intraday duty cycle transfer function 253 automatically based on this measured information.

The patient's seizure rates may also be affected by the patient's level of activity. For example, higher intensity cardio and strength training may result in fewer seizures while exercising. Similarly, patients who do manual labor may experience variation in the intensity of their work from day to day and/or throughout the workday. The controller 220 may be configured to adjust (e.g., decrease) the output of the intraday duty cycle transfer function 253 corresponding to the patient's active time based on the level of the patient's activity. The patient may transmit information about his or her activity level (e.g., current activity level or anticipated activity level) to the IMD 100 via, for example, the external device 114. In some examples, the IMD 100 may be configured to measure the patient's level of activity, and the controller 220 may be configured to adjust the intraday duty cycle transfer function 253 automatically based on this measured information.

Changes in the time of day resulting from daylight savings changes (e.g., shifting the time forward or backwards by an hour) may also affect the patient's intraday seizure rate patterns by, for example shifting the patient's seizure rate pattern forward or backwards by an hour. The controller 220 may be configured to time-shift the patient's intraday duty cycle transfer function 253 by an hour for one or more days following a daylight savings time change event (e.g., following a daylight savings time shift in the IMD's 100 internal clock 212). The controller 220 may be configured to reverse this shift after the one or more days to account for the patient's body adjusting to the new time schedule. The controller 220 may be configured to perform these adjustments to the intraday duty cycle transfer function 253 automatically or in response to the IMD 100 receiving instructions or information relating to a daylight savings time change event, for example, from the external device 114.

The patient's seizure rates may also be affected by changes in the environmental factors resulting from the seasons of the year. For example, the seizure rate in the patient may be affected, directly or indirectly, by temperature, humidity, and/or hours of sunlight, which may vary throughout the year. The controller 220 may be configured to adjust one or both of the intraday and multi-day duty cycle transfer functions 253 and 254 based on the day of the year to account for seasonal changes in these environmental factors.

The seizure rates in a biological woman may be affected by her ovulation schedule. The controller 220 may be configured to adjust the outputs of one or both of the intraday and multi-day duty cycle transfer functions 253 and 254 based on the onset of ovulation. The controller 220 may be configured to estimate the expected time of ovulation, based on the patient's historical ovulation cycle, and to automatically implement adjustments. In some other examples, the controller 220 may be configured to implement these adjustments in response to receiving (e.g., from the external device 114) information about the onset of the patient's ovulation cycle.

A patient's seizure rates may be affected by other activity, environmental, and biological factors, and the controller 220 may be configured to adjust one or both of the intraday and multi-day duty cycle transfer functions 253 and 254 based on changes in such factors.

Examples of generating duty cycle transfer functions have been described herein. Such duty cycle transfer functions may be generated automatically, for example, via the controller 220 without assistance from the patient or physician. In some other examples, these duty cycle transfer functions may be manually generated or selected by the patient or physician manually transmitting (e.g., via the external device 114) instructions to the IMD 100. For example, the patient or physician may select a suitable duty cycle transfer function from a plurality of options, or controllably select and/or adjust individual outputs or parameters of the duty cycle transfer function.

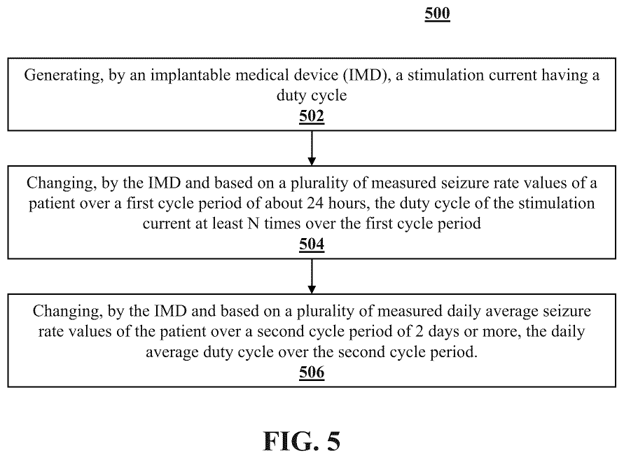

FIG. 5 depicts a method 500 for operating an implantable medical device (IMD) according to some embodiments. The IMD may be, for example, any IMD described herein or within the scope of the present disclosure. The method 500 may include a first operation 502 of generating, by the IMD, a stimulation current having a duty cycle.

During a second operation 504, the IMD may change, based on a plurality of measured seizure rate values of a patient over a first cycle period of about 24 hours (e.g., within 5%, 1%, or 0.1% of exactly 24 hours), the duty cycle of the stimulation current at least N times over the first cycle period. The plurality of measured seizure rate values may respectively correspond to a plurality of intraday time periods that span at least part of the first cycle period. The number (N) of times that the duty cycle is changed may be 3, 4, 6, 8, 12, 24 times or more and may correspond to (e.g., be equal to) the number of the plurality of measured seizure rate values. For example, the duty cycle may change at each of the plurality of intraday time periods according to the corresponding number of measured seizures during that intraday time period. In some examples, the number (N) of times that the duty cycle changes may be greater than or less than the number of the plurality of intraday time periods. The duty cycle may be changed over the first cycle period in accordance with an intraday duty cycle transfer function, for example, any intraday duty cycle transfer function described herein or within the scope of the present disclosure.

During a third operation 506 of the method 500, the IMD may change, based on a plurality of measured daily average seizure rate values of the patient over a second cycle period of 2 days or more, the daily average duty cycle over the second cycle period. For example, the duty cycle may be changed such that the daily average duty cycle changes from day-to-day based on the corresponding measured daily average seizure rate values. The second cycle period may encompass the first cycle period and, in some examples, be an integer multiple of the first cycle period. The duty cycle may be changed over the second cycle period in accordance with a multi-day duty cycle transfer function, for example, any multi-day duty cycle transfer function described herein or within the scope of the present disclosure.

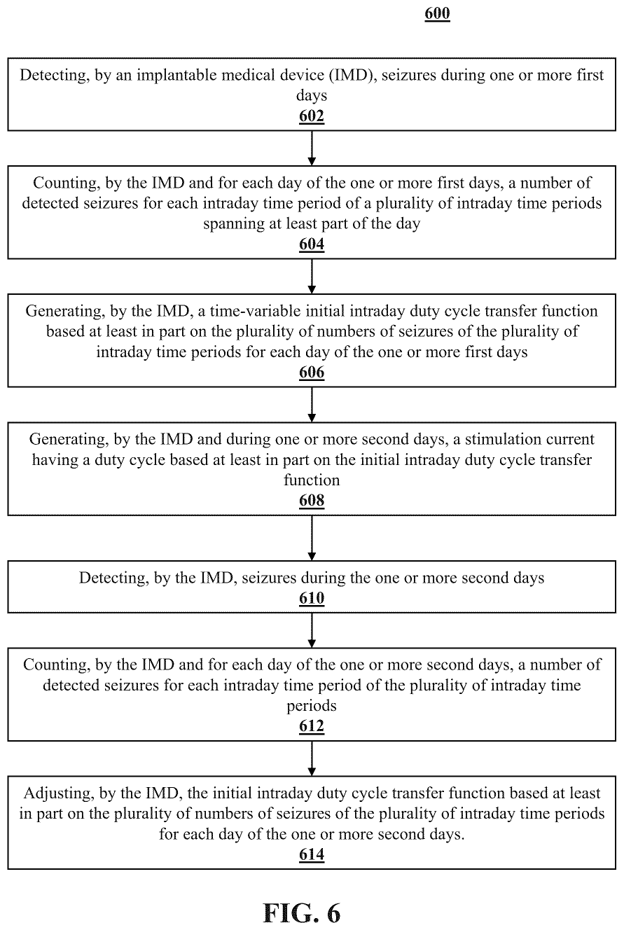

FIG. 6 depicts a method 600 for generating, by an implantable medical device (IMD), an intraday duty cycle transfer function according to some examples. The IMD may be any IMD described herein or within the scope of the present disclosure. The intraday duty cycle transfer function may be any intraday duty cycle transfer function described herein or within the scope of the present disclosure.

During a first operation 602, the IMD may detect seizures in a patient during one or more first days. In some examples, no electrical stimulation is provided to the patient during the one or more first days. In some other examples, electrical stimulation may be provided.

During a second operation 604, the IMD may count, for each day of the one or more first days, a number of detected seizures for each intraday time period of a plurality of intraday time periods spanning at least part of the day. In some other examples, the seizures may be detected and/or counted by a separate sensor device, and information about the number of counted seizures may be transmitted to the IMD.

During a third operation 606, the IMD may generate a time-variable initial intraday duty cycle transfer function based at least in part on the plurality of numbers of seizures of the plurality of intraday time periods for each day of the one or more first days. The intraday duty cycle transfer function may be generated in any manner described herein or within the scope of the present disclosure.

During a fourth operation 608, the IMD may generate, during one or more second days after the one or more first days, a stimulation current having a duty cycle based at least in part on the initial intraday duty cycle transfer function. This stimulation current may be used to treat the patient to reduce the occurrence and severity of seizures. For example, the stimulation current may be provided to the patient's vagus nerve.

During a fifth operation 610, the IMD may detect seizures during the one or more second days.

During a sixth operation 612, the IMD may count, for each day of the one or more second days, a number of seizures detected for each intraday time period of the plurality of intraday time periods. These intraday time periods may be the same intraday time periods as described with reference to operation 604.

During a seventh operation 614, the IMD may adjust the initial intraday duty cycle transfer function based at least in part on the plurality of numbers of seizures of the plurality of intraday time periods for each day of the one or more second days. This adjustment may be in any manner described herein or within the scope of the present disclosure.

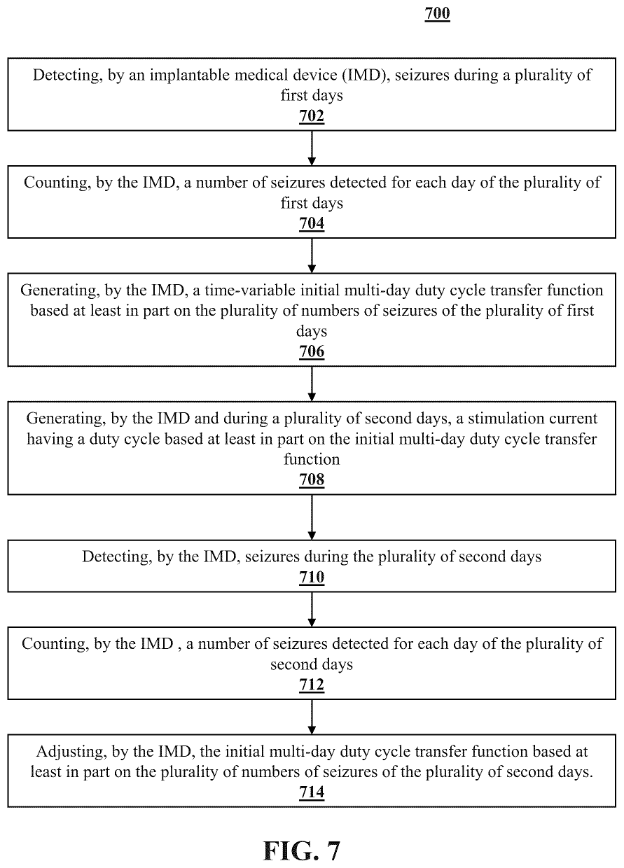

FIG. 7 depicts a method 700 for generating, by an implantable medical device (IMD), a multi-day duty cycle transfer function according to some examples. The IMD may be any IMD described herein or within the scope of the present disclosure. The multi-day duty cycle transfer function may be any multi-day duty cycle transfer function described herein or within the scope of the present disclosure.

During a first operation 702, an implantable medical device (IMD) may detect seizures in a patient during a plurality of first days. In some examples, no electrical stimulation is provided to the patient during the plurality of first days. In some other examples, electrical stimulation may be provided.

During a second operation 704, the IMD may count a number of seizures detected on each day of the plurality of first days. In some other examples, the seizures may be detected and counted by a separate sensor device, and information about the number of counted seizures may be transmitted to the IMD.

During a third operation 706, the IMD may generate a time-variable initial multi-day duty cycle transfer function based at least in part on the plurality of numbers of seizures of the plurality of first days. The multi-day duty cycle transfer function may be generated in any manner described herein or within the scope of the present disclosure.

During a fourth operation 708, the IMD may generate, during a plurality of second days after the plurality of first days, a stimulation current having a duty cycle based at least in part on the initial multi-day duty cycle transfer function. This stimulation current may be used to treat the patient to reduce the occurrence and severity of seizures. For example, the stimulation current may be provided to the patient's vagus nerve.

During a fifth operation 710, the IMD may detect seizures during the plurality of second days.

During a sixth operation 712, the IMD may count a number of seizures detected for each day of the plurality of second days.

During a seventh operation 714, the IMD may adjust the initial multi-day duty cycle transfer function based at least in part on the plurality of numbers of seizures of the plurality of second days. This adjustment may be in any manner described herein or within the scope of the present disclosure.

Although nonlimiting and non-exhaustive example methods for generating duty cycle transfer functions and/or for providing a stimulation current have been described with reference to FIGS. 5-7, the present disclosure is not limited thereto. Nonlimiting and non-exhaustive example systems for generating duty cycle transfer functions and/or for providing a stimulation current, and processes performed by such systems, have been described herein with reference to FIGS. 1-4. The present disclosure encompasses all methods for generating duty cycle transfer functions and/or for providing a stimulation current that include any combination of such processes in any suitable order.

Features from an embodiment, or from multiple embodiments, described in the present disclosure may be combined with each other, partially or entirely, and may be technically interlocked and operated in various ways, and the embodiments described herein may be implemented independently of each other or in conjunction with each other.

Although specific embodiments are described herein, the scope of the technology is not limited to those specific embodiments. Moreover, while different embodiments may be described separately, such embodiments and examples may be combined with one another in implementing the technology described herein. One skilled in the art will recognize other embodiments or improvements that are within the scope and spirit of the present technology. Therefore, the specific elements, features, and processes are disclosed only as example embodiments. The scope of the technology is defined by the following claims and any equivalents thereof.

What is claimed is:

1. A medical device comprising an implantable pulse generator (IPG), the IPG comprising:
a driver configured to generate a stimulation current having a duty cycle;
a memory storing instructions; and
a microcontroller circuit operatively coupled to the driver and to the memory, and configured, in response to executing the instructions stored in the memory, to cause the driver to vary the duty cycle based at least in part by a time-variable primary duty cycle transfer function.

2. The medical device of claim 1, wherein the primary duty cycle transfer function is at least partly based on a time-variable intraday duty cycle transfer function.

3. The medical device of claim 2, wherein, for each intraday time period of a plurality of distinct intraday time periods that collectively span a cycle period of about 24 hours, the intraday duty cycle transfer function outputs a corresponding duty cycle value during the intraday time period, and
wherein the intraday duty cycle transfer function is cyclical over the cycle period.

4. The medical device of claim 3, wherein the plurality of intraday time periods are equal to each other and comprise at least 4 intraday time periods.

5. The medical device of claim 3, wherein the intraday duty cycle transfer function is defined based at least in part on a plurality of seizure rate values respectively corresponding to the plurality of intraday time periods.

6. The medical device of claim 5, wherein the medical device is configured to detect seizures and to count the plurality of seizure rate values.

7. The medical device of claim 2, wherein the intraday duty cycle transfer function is defined based at least in part on a plurality of seizure rate values respectively corresponding to a plurality of intraday time periods that collectively span at least part of a cycle period equal to about 24 hours, each of the plurality of intraday time periods being equal to or less than 6 hours.

8. The medical device of claim 2, wherein the primary duty cycle transfer function is further based at least in part on a time-variable multi-day duty cycle transfer function.

9. The medical device of claim 8, wherein the primary duty cycle transfer function comprises a scalar product of the intraday duty cycle transfer function and the multi-day duty cycle transfer function, wherein the multi-day duty cycle outputs a scalar value based on what day it is.

10. The medical device of claim 8, wherein the multi-day duty cycle transfer function is cyclical over a period equal to or greater than two days.

11. The medical device of claim 1, wherein the primary duty cycle transfer function is based at least in part on a time-variable multi-day duty cycle transfer function.

12. The medical device of claim 11, wherein the multi-day duty cycle transfer function is cyclical over a period equal to or greater than two days.

13. The medical device of claim 11, wherein the multi-day duty cycle transfer function is defined at least in part on a plurality of seizure rate values respectively corresponding to a plurality of days.

14. The medical device of claim 11, wherein, for each day of a plurality of days, the multi-day duty cycle transfer function outputs a corresponding value.

15. The medical device of claim 14, wherein the output values of the multi-day duty cycle transfer function respectively for the plurality of days are based on a corresponding plurality of daily seizure rates.

16. The medical device of claim 15, wherein the medical device is configured to detect seizures, and to count the plurality of daily seizure rates.

17. The medical device of claim 1, wherein the primary duty cycle transfer function is based at least in part on a plurality of seizure rate that respectively correspond to a plurality of time periods.

18. The medical device of claim 17, wherein the medical device is configured to detect seizures of a person that the medical device is implanted in, and to count the plurality of seizure rates.

19. The medical device of claim 18, wherein the microcontroller circuit is configured to generate the duty cycle transfer function based at least in part on the plurality of seizure rate values.

20. The medical device of claim 17, wherein the plurality of time periods comprise a plurality of intraday time periods that collectively span at least part of a cycle period equal to about 24 hours.

21. The medical device of claim 20, wherein the plurality of intraday time periods comprise at least 4 intraday time periods.

22. The medical device of claim 17, wherein the plurality of time periods comprise at least two periods, each of about 24 hours.

23. The medical device of claim 1, wherein the microcontroller circuit is configured to time-shift, or adjust, the duty cycle transfer function.

24. The medical device of claim 23, wherein the primary duty cycle transfer function is based at least in part on a time-variable intraday duty cycle transfer function, and the microcontroller circuit is configured to time-shift, or adjust, the intraday duty cycle transfer function.

25. The medical device of claim 24, wherein the microcontroller circuit is configured to time-shift, or adjust, the intraday duty cycle transfer function based on data about at least one of a daylight savings time change, a person's sleep schedule for a previous night, the person's quality of sleep for the previous night, the person's activity schedule during the day, or the level of activity of the person during the day.

26. The medical device of claim 23, wherein the primary duty cycle transfer function is based at least in part on a time-variable multi-day duty cycle transfer function, and the microcontroller circuit is configured to time-shift, or adjust, the multi-day duty cycle transfer function.

27. The medical device of claim 26, wherein the microcontroller circuit is configured to time-shift, or adjust, the multi-day duty cycle transfer function based on data about at least one of a person's ovulation schedule or a time of year.

28. The medical device of claim 1, comprising:
A stimulation lead electrically coupled to the IPG; and
a stimulation electrode on the stimulation lead and configured to provide electrical stimulation to tissue around the stimulation electrode,
wherein the driver is configured to provide the stimulation electrode to the stimulation electrode through the stimulation lead.

29. The medical device of claim 28, wherein the stimulation electrode is configured to stimulate a vagus nerve.

30. A medical device comprising an implantable pulse generator (IPG), the IPG comprising:
a driver configured to generate a stimulation current having a duty cycle;
a memory storing instructions; and
a microcontroller circuit operatively coupled to the driver and to the memory, and configured, in response to executing the instructions stored in the memory, to cause the driver to vary the duty cycle in accordance with a primary duty cycle transfer function that is based at least in part on a plurality of measured seizure rate values respectively corresponding to a plurality of time periods.

31. The medical device of claim 30, wherein the primary duty cycle transfer function comprises an intraday duty cycle transfer function that outputs a duty cycle value that varies throughout a cycle period of about 24 hours.

32. The medical device of claim 31, wherein the plurality of time periods collectively span at least part of the cycle period, and
wherein, during each of the plurality of time periods, the intraday duty cycle transfer function outputs a duty cycle value based on the corresponding seizure rate value.

33. The medical device of claim 31, wherein the plurality of time periods collectively span at least part of the cycle period and comprise at least 4 time periods.

34. The medical device of claim 30, wherein the primary duty cycle transfer function comprises a multi-day duty cycle transfer function that varies throughout a cycle period of two days or more.

35. The medical device of claim 34, wherein the plurality of time periods collectively span at least part of the cycle period and are each about 24 hours.

36. The medical device of claim 35, wherein, during each of the plurality of time periods, the multi-day duty cycle transfer function outputs a single value based on the corresponding seizure rate value.

37. The medical device of claim 30, wherein the primary duty cycle transfer function comprises both an intraday duty cycle transfer function and a multi-day duty cycle transfer function, the intraday duty cycle transfer function varies throughout a first cycle period of about 24 hours, and the multi-day duty cycle transfer function varies throughout a second cycle period of two days or more.

38. The medical device of claim 37, wherein the plurality of time periods comprise a first set of time periods and a second set of time periods, the first set of time periods collectively span at least part of the first cycle period and comprise at least 4 time periods, and the second set of time periods collectively span at least part of the second cycle period and are each about 24 hours.

39. The medical device of claim 37, wherein the primary duty cycle transfer function is defined at least in part by a scalar product of the intraday duty cycle transfer function and the multi-day duty cycle transfer function,
wherein, for each time period of the first set of time periods, the intraday duty cycle transfer function outputs a duty cycle value during the time period based on the corresponding seizure rate value,
wherein, for each time period of the second set of time periods, the multi-day duty cycle transfer function outputs a scalar value based on the corresponding seizure rate value.

40. The medical device of claim 30, wherein the medical device is configured to detect seizures, and to count the plurality of seizure rate values.

41. The medical device of claim 40, wherein the microcontroller circuit is configured to generate the primary duty cycle transfer function based at least in part on the plurality of seizure rate values.

42. The medical device of claim 41, wherein the generating the duty cycle transfer function comprises identifying one or more cyclical patterns in the plurality of seizure rate values with respect to their corresponding plurality of time periods.

43. The medical device of claim 30, wherein the microcontroller circuit is configured to time-shift, or adjust, the duty cycle transfer function.

44. The medical device of claim 43, wherein the microcontroller circuit is configured to time-shift, or adjust, the duty cycle transfer function based on data about at least one of a daylight savings time change, a person's sleep schedule for a previous night, the person's quality of sleep for the previous night, the person's activity schedule during the day, the level of activity of the person during the day, the person's ovulation schedule, or a time of year.

45. The medical device of claim 30, comprising:
a stimulation lead electrically coupled to the IPG; and
a cuff electrode on the stimulation lead and configured to provide electrical stimulation to a vagus nerve,
wherein the driver is configured to provide the electrical current to the cuff electrode through the stimulation lead.

46. A method of operating a medical device, the medical device comprising an implantable pulse generator (IPG), the method comprising:
generating, by the IPG, a stimulation current having a duty cycle; and
varying the duty cycle based on a plurality of seizure rate values respectively corresponding to a plurality of time periods.

47. The method of claim 46, wherein the varying the duty cycle comprises varying the duty cycle over a first period of about 24 hours.

48. The method of claim 47, wherein the varying the duty cycle comprises changing the duty cycle at least 4 times over the first period.

49. The method of claim 47, wherein the duty cycle is varied based on a cyclical intraday duty cycle transfer function.

50. The method of claim 47, wherein the varying the duty cycle comprises varying the daily average duty cycle over a second period of two days or more, the second period comprising the first period.

51. The method of claim 47, wherein the plurality of time periods comprise at least 4 intraday time periods that collectively span at least part of the first period, and wherein the varying the duty cycle comprises, during each of the at least 4 intraday time periods, generating the stimulation current with a duty cycle that is based on the corresponding seizure rate value.

52. The method of claim 46, wherein the varying the duty cycle comprises varying the daily average duty cycle over a second period of at least two days.

53. The method of claim 52, wherein the duty cycle is varied in accordance with a cyclical multi-day duty cycle transfer function.

54. The method of claim 52, wherein the plurality of time periods comprise at least two daylong time periods, each being about 24 hours, and
wherein the varying the duty cycle comprises, for each of the at least two daylong time periods, generating the stimulation current with a duty cycle that has an average over the daylong time period that is based on the corresponding seizure rate value.

55. The method of claim 46, comprising measuring, by the IPG, the plurality of seizure rate values.

56. The method of claim 55, comprising generating, by the IPG, a duty cycle transfer function based on the plurality of seizure rate values,
wherein the varying the duty cycle is based at least in part on the generated duty cycle transfer function.

57. The method of claim 46, wherein the medical device further comprises a stimulation lead electrically coupled to the IPG and a cuff electrode on the stimulation lead and configured to stimulate a vagus nerve.
